(19) United States
(12) Patent Application Publication
(10) Pub. No.: US 2024/0167923 A1
(43) Pub. Date: May 23, 2024

(54) Bioengineered and standardized Human Tissue Reference Controls for Validation of IHC, FISH or CISH Cancer Test Results

(71) Applicant: SLMP, LLC, McKinney, TX (US)

(72) Inventor: Syed Ashraf IMAM, North Hollywood, CA (US)

(73) Assignee: SLMP, LLC, McKinney, TX (US)

(21) Appl. No.: 18/219,342

(22) Filed: Jul. 7, 2023

Related U.S. Application Data
(60) Provisional application No. 63/359,735, filed on Jul. 8, 2022.

(51) Int. Cl. G01N 1/36; C12M 1/00; C12N 5/077; G01N 1/28; G01N 33/574

(52) U.S. Cl. CPC G01N 1/36; C12M 23/14; C12N 5/0652; G01N 33/574; C12N 2502/30; C12N 2513/00; G01N 2001/2873; G01N 2001/368; G01N 2333/475; G01N 2474/20

(57) ABSTRACT

A method of making a tissue reference control block is disclosed wherein cancer cells are selected with a known positivity or negativity for a specific marker and cancer cells are grown to a certain phase. The cancer cells are screened for an expression of the specific marker and the screened cancer cells are mixed with stromal cells and co-cultured in a bio-reactor bag. A spheroid grown from the co-cultured mixture of cells is harvested, fixed, and embedded in a core of a paraffin block.

### BACKGROUND

Pathology testing and clinical laboratory testing are important aspects of modern diagnostic, prognostic and therapeutic-guiding practices. Reference Controls are used to maintain quality control (QC) for reproducibility and validation of test results by Immunohistochemical (IHC) staining method, Fluorescence in situ hybridization (FISH), Chromogenic in situ hybridization (CISH), and other methods of molecular tests.

Human tissue have been traditionally used as reference controls for the validation of test results for the positivity of diagnostic, prognostic, or therapeutic-guiding markers' profile on patient's tissue samples by IHC, FISH or CISH tests. Pathologists compare a known positivity profile of marker in the reference control tissue with that of the test tissue specimens from the cancer patients to deliver the clinical decision. However, a marker's positivity and its expression profile in tissue reference controls are known to vary due to a phenomenon called ‘pre-analytical variables’. The variables are introduced during the preparation by using different protocols for the fixation and processing of tissue at different laboratories, hence an intrinsic problem of not representing standardized reference controls within and between testing laboratories. Use of such reference controls has been widely reported to cause inconsistent test results which are reported to range between 10 to 30%, hence making them unreliable source of reference control for making a clinical decision to benefit the cancer patients. See e.g., Bogen, S. A. (conducting a root cause analysis into the high error rate in clinical immunohistochemistry) Appl Immunohistochem Mol Morphol, Volume 27, Number 5, pages 329-338, 2019, and Kirbis, I. S. and Kholova, I., discussing nationwide differences in cytology fixation and processing methods and their impact on interlaboratory variation in PD-L1 positivity, Virchows Archiv, Volume 482, pages 797-798, 2023. Moreover, tissue reference controls with a known marker's profile are not readily available in large enough quantities to serve as the standardized reference controls for the validation of test results in the above-stated test, particularly within or in between large pathologic testing laboratories nationwide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Illustrative embodiments of the present invention are described in detail below with reference to the attached drawing figures, which are incorporated by reference herein, and wherein:

FIG. 1A illustrates an image of a bioengineered human tissue control formed by the processes described herein and stained to illustrate the presence of Her-2/neu marker at a high level (3+) of expression and in accordance with one embodiment.

FIG. 1B illustrates an image of actual human tissues stained to illustrate the presence of Her-2/neu marker at a high level (3+) of expression and in accordance with one embodiment.

FIG. 1C illustrates an image of a human cell line control stained to illustrate the presence of Her-2/neu marker at a high level (3+) of expression and in accordance with one embodiment.

FIG. 1D illustrates another image of a bioengineered human tissue control formed by the processes described herein and stained to illustrate the presence of Her-2/neu marker at a medium level (2+) of expression and in accordance with one embodiment.

FIG. 1E illustrates another image of actual human tissues stained to illustrate the presence of Her-2/neu marker at a medium level (2+) of expression and in accordance with one embodiment.

FIG. 1F illustrates another image of a bioengineered human tissue control formed by the processes described herein to illustrate the absence of Her-2/neu marker expression in accordance with one embodiment.

FIG. 1G illustrates another image of actual human tissues stained to illustrate the absence of Her-2/neu marker expression in accordance with one embodiment.

FIG. 1H illustrates another image of a human cell line control stained to illustrate the absence of Her-2/neu marker expression in accordance with one embodiment.

FIG. 2A illustrates an image of a bioengineered human tissue control formed by the processes described herein and stained to illustrate the presence of p16 at a high level (3+) of expression in accordance with one embodiment.

FIG. 2B illustrates an image of actual human tissues stained to illustrate the presence of p16 at a high level (3+) of expression in accordance with one embodiment.

FIG. 3A illustrates an image of a bioengineered human tissue control formed by the processes described herein and stained to illustrate the presence of p53 at a high level (3+) of expression in accordance with one embodiment.

FIG. 3B illustrates an image of actual human tissues stained to illustrate the presence of p53 at a high level (3+) of expression in accordance with one embodiment.

FIG. 4A illustrates an image of a bioengineered human tissue control formed by the processes described herein and stained to illustrate the presence of ki-67 at a high level (3+) of expression in accordance with one embodiment.

FIG. 4B illustrates an image of actual human tissues stained to illustrate the presence of ki-67 at a high level (3+) of expression in accordance with one embodiment.

FIG. 5A illustrates an image of a bioengineered human tissue control formed by the processes described herein and stained to illustrate the presence of PD-L1 at a high level (3+) of expression in accordance with one embodiment.

FIG. 5B illustrates an image of actual human tissues stained to illustrate the presence of PD-L1 at a high level (3+) of expression in accordance with one embodiment.

FIG. 6A illustrates an image of a bioengineered human tissue control formed by the processes described herein and stained to illustrate the presence of GATA-3 at a high level (3+) of expression in accordance with one embodiment.

FIG. 6B illustrates an image of actual human tissues stained to illustrate the presence of GATA-3 at a high level (3+) of expression in accordance with one embodiment.

The illustrated figures are only exemplary and are not intended to assert or imply any limitation with regard to the environment, architecture, design, or process in which different embodiments may be implemented.

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments is defined only by the appended claims.

The present disclosure relates to bioengineered human tissue reference controls. More particularly, bioengineered human tissue controls, and systems and methods to form and utilize bioengineered human tissue reference are disclosed. Additional descriptions of processes to form human tissue reference controls can be found in U.S. Pat. No. 10,329,623. In some embodiments, a process to utilize bioengineered human tissue reference controls include one or more of the following steps:

(1) Growing Cancer Cells in a Specific Phase:

Cancer cells with known positivity or negativity for a specific marker are identified and grown in culture. In some embodiments, the cancer cells are grown in an S phase or mostly S phase in culture before they are harvested. In some embodiments, the cancer cells are grown to a G2M phase in culture before they are harvested. In some embodiments, cancer cells are grown into another phase that produces cancer cells having a desired positivity or negativity for a marker and production of IHC standardized reference controls. In some embodiments, cancer cells are grown into another phase that produces cancer cells having a consistently known level of markers' expression at a high: 3+, medium: 2+, or negative: 0 across multiple batches of controls subsequently formed from the cancer cells to eliminate antigenic drift, where a consistent high level, medium level, or negative refer to 3+, 2+, and 0 level of expression, respectively, are maintained.

(2) Pre-Screening of Cancer Cells with Positivity for a Marker:

An aliquot of the cells in their growth phases (S and G2M) is harvested, placed on a histologic glass slide, fixed in 10% neutral buffered formalin (NBF), washed in Dulbecco phosphate buffered saline (dPBS), and screened for the expression of a marker using a specific antibody by a standard IHC staining method. In some embodiments, such a strict criteria for harvesting cells is used to optimize the expression of the markers of cancer cells is applied to eliminate antigenic drift, which criteria is known to have negative impact on the level of markers ‘expression. In some embodiments, the specific criteria for harvesting cells are determined based on a specific marker of interest, and by the IHC staining for the specific marker.

(3) Selection of Cells for Co-Culture:

In some embodiments, and in response to the result of staining of the cells, cells with a specific level of a marker's expression (e.g., high: 3+, medium 2+, or negative: 0) are used in the co-culture. In some embodiments, the results of the staining of the cells are as expected or are within a threshold range if the results are at least 90% as expected. In some embodiments, the results of the staining of the cells are as expected or are within a threshold range if the results are at least 80% as expected. Otherwise, the cancer cells are grown in the presence of known growth factor as supplements (e.g., epidermal growth factor, insulin, or insulin-like-growth factor, depending upon the cell-type) which are known to induce the expression level of the markers, hence referred to as the treated cells.

(4) Co-Culture Protocol:

Untreated or treated cells are mixed with stromal cells and co-cultured. In one embodiment, the cells are mixed in or transferred to a bag designed to grow cells and placed in a bioreactor incubator in a control environment at about 37 degrees Celsius, with about 5% carbon dioxide and humidity. In one embodiment, 3 million untreated or treated cancer cells are mixed with 120 million or approximately 120 million stromal cells. In another alternative embodiment, 4 million untreated or treated cancer cells are mixed with 120 million or approximately 120 million stromal cells. In yet another embodiment, 5 million untreated or treated cancer cells (based on the cell type) are mixed with 120 million or approximately 120 million stromal cells. In some embodiments, a higher count of stromal cells (such as over 130 million stromal cells) is used to improve the imitation or replication of actual human tissue-like morphology. In some embodiments, a higher count of stromal cells (such as over 140 million stromal cells) is used to derive additional extracellular matrix, which cancer cells invade and replicate.

(5) Duration of the Co-Culture:

In one embodiment, the duration of co-culture is 10 days to improve cell viability and tissue-like morphology of the control. In other embodiments, the duration may be shortened or extended.

(6) Harvesting of the Co-Cultured Product:

Upon the completion of the co-culture, the product is harvested from a bioreactor bag. In some embodiments, spheroids consisting of both the cancer and stromal cells are separated from that of the non-spheroid single cell (hereafter referred to as “single cell”) population to improve the quality of the standardized reference controls. In one or more of such embodiments, the spheroids containing both the cancer and stromal cells are separated from that of the single cell population. In some embodiments, the separation is achieved by four to ten rounds of washing, depending on the type of the cancer cell, and by centrifugation at 200 g and for a period of approximately 5 minutes. In one or more of such embodiments, the effectiveness of the washing process is monitored during the foregoing process. In one or more of such embodiments, a different number of rounds of washing are performed until the number of single cells is less than a threshold, such as where at least 90% (or another threshold) of the product represents the spheroid. For example, after one round of washing, the ratio of single cell to spheroid is 90:10, after a second round of washing the ratio of single cell to spheroid is 70:30, and additional rounds of washings are performed until the ratio of single cell to spheroid is 5:95, less than 5:95, or another threshold value.

(7) Construction of the Bioengineered and Standardized Human Tissue Reference Control Blocks:

The pure population (at least 95%) of the spheroids are fixed in 10% NBF for 24 hours or approximately 24 hours. In some embodiments, the fixed spheroids are embedded in a paraffin block, with the core measuring approximately 45 to 56 mm length, depending upon the cell type. In one or more of such embodiments, one or more 3 mm long segments are obtained from the paraffin block (donor) and placed into a recipient core (2 mm diameter and 3 mm depth) of a paraffin tissue microarray (TMA) block (recipient TMA block). In some embodiments, 15 (1-core), 7 (2-core) or 5 (3-core) TMA blocks are obtained per batch of a bioreactor bag. In some embodiments, segments from multiple donor blocks representing different cell types may be placed into recipient cores in the TMA block.

(8) Quality Control (QC) Test 1:

The first section (4 micron-thick) is cut from the top of each TMA block containing either 1, 2, or 3 cores (with different cell types) and placed on the positively charged histologic glass slide. In some embodiments, the slide is immunostained with a specific antibody to confirm the presence of the marker's known expression profile. In one or more of such embodiments, a positive test result ensures the presence of the cells with known expression profile of a marker in each block. See, e.g., (001); (003); (005); (007); (009). For example, a positive test result is for cells with a known positive expression profile.

(9) Quality Control (QC) Test 2:

One block per batch is cut through to its entirety and each resulting section is placed on positively charged histologic glass slides and immunostained as stated above. A positive test result ensures the presence of the cells with required expression profile of a marker in every section from the top to the bottom of the block. Typically, 450 sections (4 micron-thick each) are obtained per block. In some embodiments, multiple iterations of the quality control tests 1 are performed sequentially before quality control 2 is performed. In some embodiments, multiple iterations of the quality control tests are concurrently performed.

In some embodiments, bioengineered and standardized human tissue reference controls formed by the processes described herein share most of the essential histologic parameters with tissue controls, such as known diagnostic, prognostic, or therapeutic-guiding markers’ expression profiles, morphology, cellular polarity, stromal components plus extracellular matrix, and known expression profiles of markers at a high level (3+) of expression, medium level (2+) of expression, or negative (0),

respectively, which are critical for the validation of IHC, FISH, or CISH test results. In some embodiments, bioengineered human tissue reference controls formed by the processes described herein was consistently expressed in every slide and block in every batch of production, and significantly outperform the human tissue or human cell line reference controls by: A. High degree of concordance (98%), B. High unequivocal results (97%), C. Presence of tumor (100%), and D. No loss of tissue during the staining (100%). In some embodiments, Coefficient of variation for Her2/neu, p53, p16, Ki-67, PD-L1, or GATA-3 expression on histologic slides measured in the range of 3.11% to 3.15% over time, below the widely accepted 5% threshold for such test reproducibility. In some embodiments, coefficient of variation for Her2/neu, p53, p16, Ki-67, PD-L1, or GATA-3 expression on histologic slides measured in the range of 3.12% to 3.19% over time. In one or more of such embodiments, bioengineered human tissue reference controls formed by the processes described herein provide a complete control over verification of IHC, FISH, or CISH test results.

In some embodiments, bioengineered human tissue reference controls formed by the processes described herein reduce or eliminate a time-consuming search to replace exhausted human tissue blocks when tissue with the required biomarker's expression profile is not available, thus allowing histotech resources to focus on improved patient care and other high-value activities in pathology laboratories. In some embodiments, bioengineered human tissue reference controls formed by the processes described herein are ready-to-use slide and block of reference controls readily accommodates the existing workflow of both low and high-volume pathology laboratories. In some embodiments, the core size of bioengineered human tissue reference controls formed by the processes described herein are optimized to be used as an ‘on-slide’ control to easily accommodate a patient's tissue test sample from a cancer patient. In one or more of such embodiments, the core size of bioengineered human tissue reference controls is 8 mm long and 2 mm wide for 3-core, 6 mm long and 2 mm wide for 2-core, or 4 mm long and 2 mm wide for 1-core. In some embodiments, bioengineered human reference tissue controls formed by the processes described herein are used as reliable reference controls (i.e., a known level of expression profile of diagnostic, prognostic or therapeutic-guiding markers, morphology, cellular polarity, stromal cells and extracellular matrix) in the above-noted assays on 4 major and independent auto-strainer systems (i.e., Leica Bond III, Roche Benchmark Ultra, DAKO Omnis, and Quantum HDx).

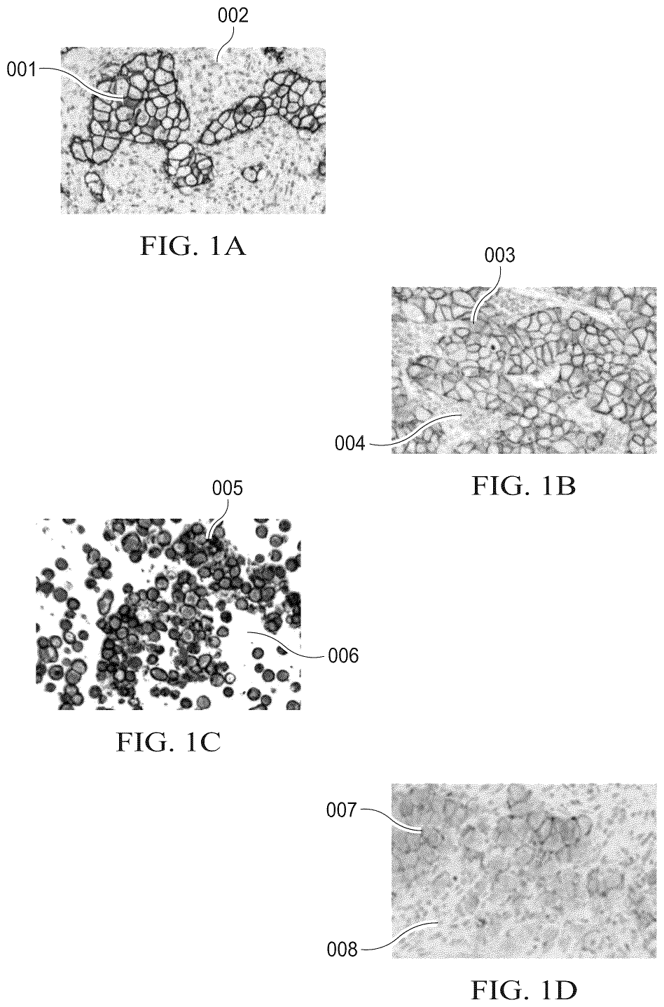

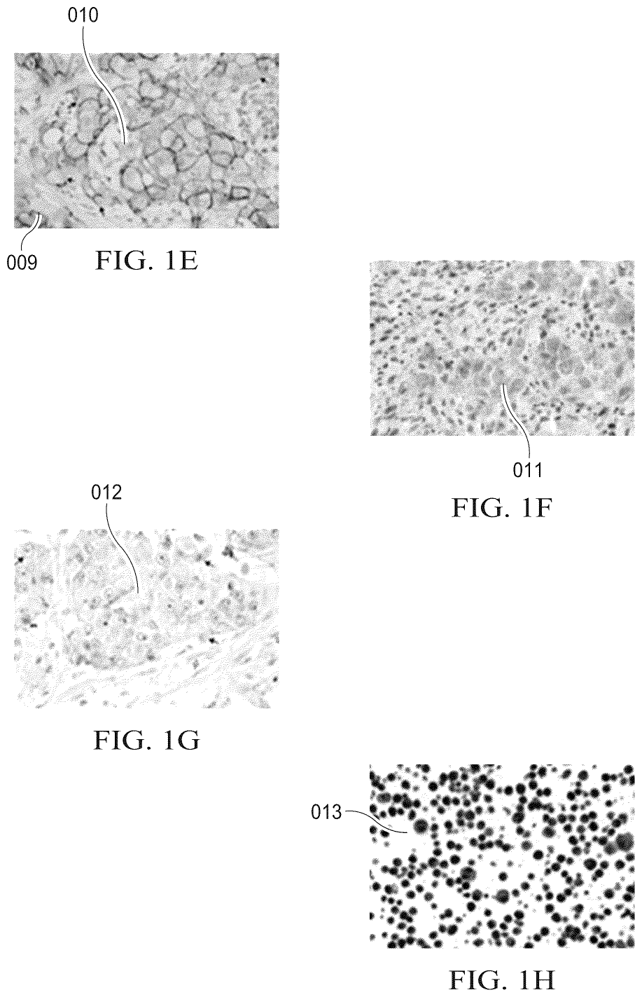

Now turning to the Figures. FIG. 1A illustrates an image of a bioengineered human tissue reference control formed by the processes described herein and stained to illustrate the presence of Her-2/neu marker at a high level (3+) of expression in accordance with one embodiment. In the embodiment of FIG. 1A, cancer cells show Her2/neu membranous staining at a high level (3+) of expression (brown) (001), whereas the stromal cells (blue nucleus) are negative (absence of brown staining) (002). FIG. 1B illustrates an image of actual human tissues stained to illustrate the presence of Her-2/neu marker at a high level (3+) of expression in accordance with one embodiment. In FIG. 1B, cancer cells show Her2/neu membranous staining at a high level (3+) of expression (brown) (003), whereas the stromal cells (blue nucleus) (004) are negative (absence of brown staining). In the embodiment of FIG. 1A, cells that are known to be cancer positive are clearly shown as positive (brown) (001) whereas cells that are known to be negative are clearly shown as negative (blue) (002). Moreover, the contrast level of positive to negative cells as illustrated in FIG. 1A are similar to the contrast level of positive to negative cells as illustrated in FIG. 1B, which indicates the similar or identical resemblances of bioengineered human tissue reference controls formed by the processes described herein relative to actual human tissues. The foregoing contrasts FIG. 1C, which illustrates an image of a human cell line reference control stained to illustrate the presence of Her-2/neu marker at a high level (3+) (005) of expression in accordance with one embodiment. More particularly, the stromal components of the human cell line control of FIG. 1C, contrary to the bioengineered human tissue reference control of FIG. 1A or the human tissues of FIG. 1B, are not visible (006), and bare little to no resemblance to the human tissue of FIG. 1B, or the bioengineered human tissue reference control of FIG. 1A that is formed by the processes described herein.

FIG. 1D illustrates another image of a bioengineered human tissue reference control formed by the processes described herein and stained to illustrate the presence of Her-2/neu marker at a medium level (2+) of expression and in accordance with one embodiment. In the embodiment of FIG. 1D, cancer cells show Her2/neu membranous staining at a medium level (2+) of expression (brown) (007), whereas the stromal cells (blue nucleus) are negative (absence of brown staining) (008). FIG. 1E illustrates another image of actual human tissues stained to illustrate the presence of Her-2/neu marker at a medium level (2+) of expression and in accordance with one embodiment. In FIG. 1E, cancer cells show Her2/neu membranous staining at a medium level (2+) of expression (brown) (009), whereas the stromal cells (blue nucleus) (010) are negative (absence of brown staining). In the embodiment of FIG. 1D, cells that are known to be cancer positive are clearly shown as positive (brown) (007) whereas cells that are known to be negative are clearly shown as negative (blue) (008). Moreover, the contrast level of positive to negative cells as illustrated in FIG. 1D are similar to the contrast level of positive to negative cells as illustrated in FIG. 1E, which indicates the similar or identical resemblances of bioengineered human tissue reference controls formed by the processes described herein relative to actual human tissues.

FIG. 1F illustrates another image of a bioengineered human tissue reference control formed by the processes described herein to illustrate the absence of Her-2/neu marker expression in accordance with one embodiment. In the embodiment of FIG. 1F, cancer cells are negative (absence of brown staining) (011). FIG. 1G illustrates another image of actual human tissues stained to illustrate the absence of Her-2/neu marker expression in accordance with one embodiment. In FIG. 1G, cancer cells are negative (absence of brown staining) (012). FIG. 1H illustrates another image of a human cell line control stained to illustrate the absence of Her-2/neu marker expression in accordance with one embodiment. More particularly, the stromal components of the human cell line control of FIG. 1H, contrary to the bioengineered human tissue reference control of FIG. 1F or the actual human tissues of FIG. 1G, are not visible (013), and bare little to no resemblance to the actual human tissue of FIG. 1G, or the bioengineered human tissue reference control of FIG. 1F that is formed by the processes described herein.

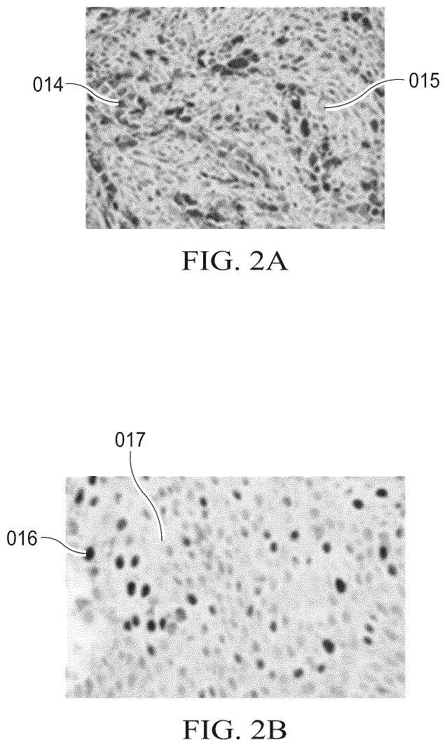

FIG. 2A illustrates an image of a bioengineered human tissue reference control formed by the processes described herein and stained to illustrate the presence of p16 at a high level (3+) of expression in accordance with one embodiment. In the embodiment of FIG. 2A, cancer cells show p16 nucleus staining at a high level (3+) of expression (brown) (014), whereas the stromal cells (blue nucleus) (015) are negative (absence of brown staining), which serves as an internal negative control for p16 expression. FIG. 2B illustrates an image of actual human tissues stained to illustrate the presence of p16 at a high level (3+) of expression in accordance with one embodiment. In the embodiment of FIG. 2B, cancer cells show p16 nucleus staining at a high level (3+) of expression (brown) (016), whereas the stromal cells (blue nucleus) are negative (absence of brown staining) (017). In the embodiment of FIG. 2A, cells that are known to be cancer positive are clearly shown as positive at a high level (3+) of expression (brown) whereas cells that are known to be negative are clearly shown as negative (blue), which serves as the internal negative control for p16 expression. Moreover, the contrast level of positive to negative cells in bioengineered human tissue reference control as illustrated in FIG. 2A are similar to the contrast level of positive to negative cells in the actual human tissue as illustrated in FIG. 2B, which indicates the similar or identical resemblances of bioengineered human tissue reference controls formed by the processes described herein relative to actual human tissues.

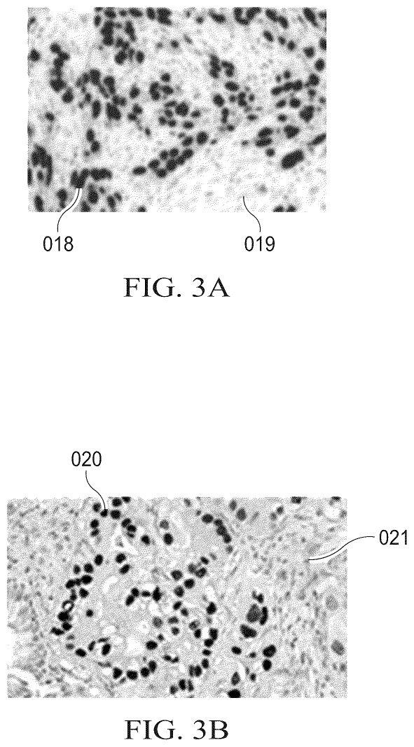

FIG. 3A illustrates an image of a bioengineered human tissue reference control formed by the processes described herein and stained to illustrate the presence of p53 at a high level (3+) of expression in accordance with one embodiment. In the embodiment of FIG. 3A, cancer cells show p53 nucleus staining at a high level (3+) of expression (brown) (018), whereas the stromal cells (blue nucleus) are negative (absence of brown staining) (019). FIG. 3B illustrates an image of actual human tissues stained to illustrate the presence of p53 expression at a high level (3+) of expression in accordance with one embodiment. In FIG. 3B, cancer cells show p53 nucleus staining at a high level (3+) of expression (brown) (020), whereas the stromal cells (blue nucleus) (021) are negative (absence of brown staining). In the embodiment of FIG. 3A, cells that are known to be cancer positive are clearly shown as positive at a high level (3+) of expression (brown) (018) whereas cells that are known to be negative are clearly shown as negative (blue) (019), which serves as the internal negative control for p53 expression. Moreover, the contrast level of positive to negative cells as illustrated in FIG. 3A are similar to the contrast level of positive to negative cells as illustrated in FIG. 3B, which indicates the similar or identical resemblances of bioengineered human tissue reference controls formed by the processes described herein relative to actual human tissues.

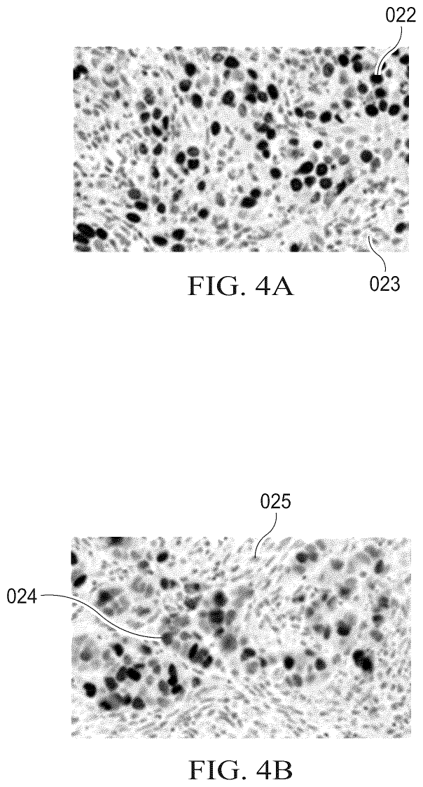

FIG. 4A illustrates an image of a bioengineered human tissue reference control formed by the processes described herein and stained to illustrate the presence of ki-67 at a high level (3+) of expression in accordance with one embodiment. In the embodiment of FIG. 4A, cancer cells show ki-67 nucleus staining at a high level (3+) of expression (brown) (022), whereas the stromal cells (blue nucleus) (023) are negative (absence of brown staining), which serves as the internal negative control for Ki-67 expression. FIG. 4B illustrates an image of actual human tissues stained to illustrate the presence of ki-67 at a high level (3+) of expression in accordance with one embodiment. In FIG. 4B, cancer cells show ki-67 nucleus staining at a high level (3+) of expression (brown) (024), whereas the stromal cells (blue nucleus) (025) are negative (absence of brown staining), which serves as the internal negative control for Ki-67 expression. Moreover, the contrast level of positive to negative cells as illustrated in FIG. 4A are similar to the contrast level of positive to negative cells as illustrated in FIG. 4B, which indicates the similar or identical resemblances of bioengineered human tissue reference controls formed by the processes described herein relative to actual human tissues.

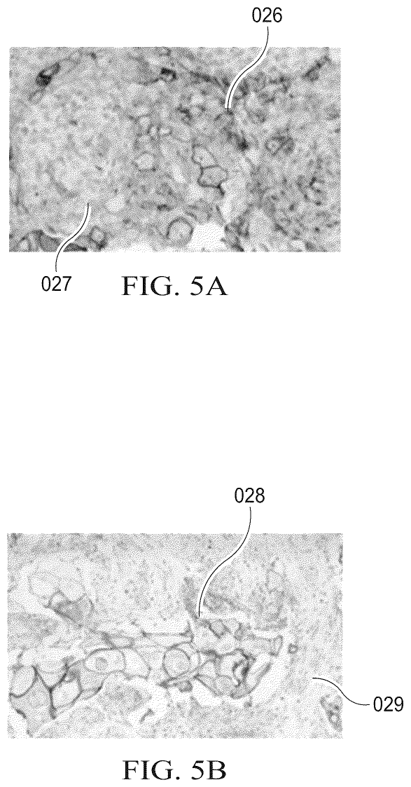

FIG. 5A illustrates an image of a bioengineered human tissue reference control formed by the processes described herein and stained to illustrate PD-L1 at a high level (3+) of expression in accordance with one embodiment. In the embodiment of FIG. 5A, cancer cells show PD-L1 membranous staining at a high level (3+) of expression (brown) (026), whereas the stromal cells (blue nucleus) (027) are negative (absence of brown staining), which serve as the internal negative control for PD-L1 expression. FIG. 5B illustrates an image of actual human tissues stained to illustrate the presence of PD-L1 at a high level (3+) of expression in accordance with one embodiment. In FIG. 5B, cancer cells show PD-L1 membranous staining at a high level (3+) of expression (brown) (028), whereas the stromal cells (blue nucleus) (029) are negative (absence of brown staining), which serve as the internal negative control for PD-L1 expression. Moreover, the contrast level of positive to negative cells as illustrated in FIG. 5A are similar to the contrast level of positive to negative cells as illustrated in FIG. 5B, which indicates the similar or identical resemblances of bioengineered human tissue reference controls formed by the processes described herein relative to actual human tissues.

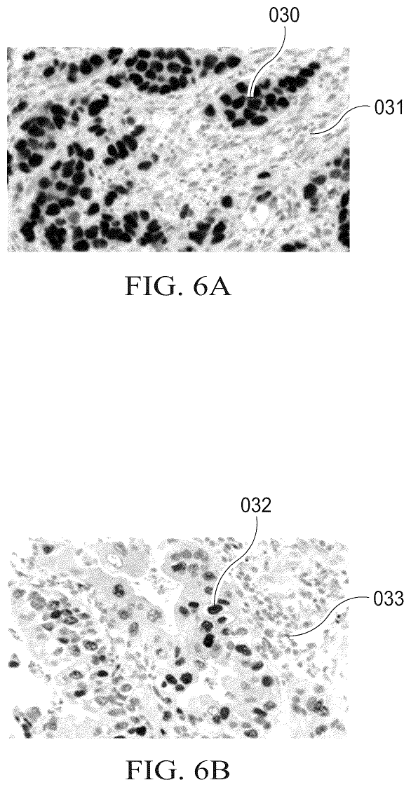

FIG. 6A illustrates an image of a bioengineered human tissue reference control formed by the processes described herein and stained to illustrate GATA-3 at a high level (3+) of expression in accordance with one embodiment. In the embodiment of FIG. 6A, cancer cells show GATA-3 nucleus staining at a high level (3+) of expression (brown) (030), whereas the stromal cells (blue nucleus) (031) are negative (absence of brown staining) which serve as the internal negative control for GATA-3 expression. FIG. 6B illustrates an image of actual human tissues stained to illustrate the presence of GATA-3 at a high level (3+) of expression in accordance with one embodiment. In FIG. 6B, cancer cells show GATA-3 nucleus staining at a high level (3+) of expression (brown) (032), whereas the stromal cells (blue nucleus) (033) are negative (absence of brown staining) which serve as the internal negative control for GATA-3). Moreover, the contrast level of positive to negative cells as illustrated in FIG. 6A are similar to the contrast level of positive to negative cells as illustrated in FIG. 6B, which indicates the similar or identical resemblances of bioengineered human tissue reference controls formed by the processes described herein relative to actual human tissues.

The above disclosed embodiments have been presented for purposes of illustration and to enable one of ordinary skill in the art to practice the disclosed embodiments but are not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification.

As used herein, the singular forms “a,” “an,” and “the” are intended to include the plural forms as well, unless the context clearly indicates otherwise. It will be further understood that the terms “comprise” and/or “comprising,” when used in this specification and/or the claims, specify the presence of stated features, steps, operations, elements, and/or components, but do not preclude the presence or addition of one or more other features, steps, operations, elements, components, and/or groups thereof. In addition, the steps and components described in the above embodiments and figures are merely illustrative and do not imply that any particular step or component is a requirement of a claimed embodiment.

What is claimed is:

1. A method of making a tissue reference control block comprising:
Selecting cancer cells with a known positivity or negativity for a specific marker;
Growing the cancer cells to a phase;
Screening the cancer cells in the phase for an expression of the specific marker;
Mixing the screened cancer cells with stromal cells;
Co-culturing the mixture of cells in a bio-reactor bag;
Harvesting a spheroid grown from the co-cultured mixture of cells;
Fixing the harvested spheroid; and
Embedding the fixed spheroid in a core of a paraffin block.

2. The method of claim 1 in which the phase is a phase that has positivity or negativity for the specific marker.

3. The method of claim 1 in which the phase is a phase that has a consistently known level of the specific marker.

4. The method of claim 3 in which the consistently known level of the specific marker is high, medium, or negative.

5. The method of claim 1 in which the expression of the specific marker is high, medium, or negative.

6. The method of claim 1 in which immunohistochemical staining is used to screen the cancer cells in the phase for an expression of the specific marker.

7. The method of claim 6 in which the expression of the specific marker is a level of high, medium, or negative.

8. The method of claim 7 in which at least about 90% or at least about 80% of the cancer cells exhibit the level.

9. The method of claim 7 in which the cancer cells are grown in the presence of a growth factor which is known to induce the level when at least about 90% or at least about 80% of the cancer cells do not exhibit the level.

10. The method of claim 1 in which the mixture of cancer cells to stromal cells consists of a ratio of about 1:40, about 1:30, or about 1:24.

11. The method of claim 1 in which the mixture of cancer cells and stromal cells consists of at least 130 million stromal cells or at least 140 million stromal cells.

12. The method of claim 1 in which the mixture of cells is co-cultured for about 10 days.

13. The method of claim 1 further comprising cutting a segment from the core and placing the segment in a core of a recipient block.

14. The method of claim 13 in which the recipient block consists of a paraffin tissue microarray.

15. The method of claim 13 further comprising cutting a section of the recipient block and testing the section for a presence of a known expression profile of the specific marker.

16. The method of claim 13 further consisting of cutting the entirety of one of the recipient blocks into sections.

17. The method of claim 16 further consisting of testing the sections for a presence of a known expression profile of the specific marker.

18. The method of claim 3 in which immunohistochemical staining is used to screen the cancer cells in the phase for an expression of the specific marker.

19. The method of claim 18 in which the expression of the specific marker is a level of high, medium, or negative.

20. The method of claim 19 in which at least about 90% or at least about 80% of the cancer cells exhibit the level.

21. The method of claim 19 in which the cancer cells are grown in the presence of a growth factor which is known to induce the level when at least about 90% or at least about 80% of the cancer cells do not exhibit the level.

22. The method of claim 3 in which the mixture of cancer cells to stromal cells consists of a ratio of about 1:40, about 1:30, or about 1:24.

23. The method of claim 3 in which the mixture of cancer cells and stromal cells consists of at least 130 million stromal cells or at least 140 million stromal cells.

24. The method of claim 3 in which the mixture of cells is co-cultured for about 10 days.

25. The method of claim 3 further comprising cutting a segment from the core and placing the segment in a core of a recipient block.

26. The method of claim 25 in which the recipient block consists of a paraffin tissue microarray.

27. The method of claim 25 further comprising cutting a section of the recipient block and testing the section for a presence of a known expression profile of the specific marker.

28. The method of claim 25 further consisting of cutting the entirety of one of the recipient blocks into sections.

29. The method of claim 28 further consisting of testing the sections for a presence of a known expression profile of the specific marker.
